(19) United States
(12) Patent Application Publication
(10) Pub. No.: US 2010/0094086 A1
(43) Pub. Date: Apr. 15, 2010

(54) MEDICO-TECHNICAL FLEXIBLE POLYMER TUBE AND METHOD OF MANUFACTURING THE SAME

(75) Inventor: Bob Konstantin, Weinheim (DE)

Correspondence Address: FOLEY & LARDNER LLP, P.O. BOX 80278, SAN DIEGO, CA, 92138-0278, US

(73) Assignee: Invendo Medical GmbH

(21) Appl. No.: 12/576,199

(22) Filed: Oct. 8, 2009

(30) Foreign Application Priority Data
Oct. 9, 2008 (DE) 10 2008 042 718.7

(51) Int. Cl. A61B 1/00; B32B 27/06

(52) U.S. Cl. 600/114; 264/171.28

(57) ABSTRACT

A medico-technical flexible polymer tube of thermoplastic or of thermoplastics, a method of manufacturing a medico-technical flexible polymer tube of thermoplastic or of thermoplastics as well as an endoscope comprising a medico-technical flexible polymer tube of thermoplastic or of thermoplastics is provided.

### CROSS-REFERENCE TO RELATED PATENT APPLICATIONS

Germany Priority Application DE 10 2008 042 718.7, filed Oct. 9, 2008 including the specification, drawings, claims and abstract, is incorporated herein by reference in its entirety.

The present invention relates to a medico-technical flexible polymer tube, a method of manufacturing a medico-technical flexible polymer tube and an endoscope for which a medico-technical flexible polymer tube is used.

Devices for introducing a medical endoscope into a body canal are described, for instance, in the German patent application DE-A-39 25 484. The devices described there permit that an endoscope is no longer pushed into the body to be examined but moves into the body by itself. For this purpose, the endoscope is equipped with an inherent drive that permits easy and quick insertion.

As such an inherent drive, for instance, also a so-called eversion tube can be used into which the endoscope shaft is introduced. Upon propulsion of the endoscope different relative movements occur. On the one hand, a relative movement occurs between the endoscope shaft and the eversion tube which are in sliding contact with each other. On the other hand, a relative movement also occurs between an inside portion and an outside portion of the unwinding eversion tube.

The current development of such devices strives to manufacture tubes that are adapted to achieve a reduced friction between an inserted eversion tube, for instance, and an endoscope shaft, between the eversion tube and a cover of the endoscope shaft as well as between individual portions of an eversion tube.

With the currently used eversion tube devices the problem arises, however, that from a particular length of the tube the resistance produced by friction becomes so high that the eversion tube cannot be introduced any further into the patient. Moreover, the problem may arise that the eversion tube forms wrinkles onto the eversion tube due to the transmission of power and for this reason, too, further propulsion is not possible.

It is the object of the invention to provide an improved medico-technical tube (hereinafter also referred to “the tube”) and/or an improved method of manufacturing a medico-technical tube as well as an improved endoscope comprising a medico-technical tube.

The object according to the invention is achieved by the combinations of features defined in the independent claims. Advantageous further developments can be inferred from the subclaims.

It is especially intended by the invention to provide a medico-technical flexible polymer tube and a method of manufacturing the same which exhibits an improved mechanical behavior during introduction into the patient. In particular, it is intended by the invention to provide a medico-technical polymer tube which is adapted to be inserted into the patient over longer distances than before as well as a method of manufacturing the same.

According to a first aspect, the invention relates to a medico-technical flexible polymer tube made of thermoplastic material, the polymer tube including at least two layers which have a different Shore hardness.

According to another aspect, for achieving the aforementioned object the present invention provides a medico-technical flexible polymer tube made of thermoplastic material, the polymer tube including one or more layers and at least one surface of the polymer tube being functionalized.

In accordance with another aspect of the invention, a medico-technical flexible polymer tube of thermoplastic material is provided which includes at least two layers having a different Shore hardness and at least one surface of the polymer tube being functionalized.

Apart from that, the invention provides a method of manufacturing such medico-technical flexible polymer tubes as well as an endoscope comprising medico-technical flexible polymer tubes.

The tubes according to the invention preferably consist of at least two layers either of one type of thermoplastic or of several types of thermoplastics. The tube is preferably obtained by extrusion or co-extrusion of the at least two layers of plastic material. The plastic material is preferably selected from thermoplastics, preferably thermoplastic polyurethane (PUR), polyvinylchloride (PVC), perfluoroethylene propylene copolymer (FEP), polyamide (PA), polyethylene (PE) and/or polytetrafluoroethylene (PTFE). A lubricant or anti-friction agent can be incorporated in the plastic material(s).

The PVC material shows the advantage that it can be sterilized by virtue of the temperature resistance (to heat and cold) by all sterilizing methods common in this technical field (steam sterilization, ethylene oxide sterilization, radiation sterilization, low-temperature plasma (sterad-plasma) sterilization) and can be easily stored. PVC moreover has a high buckling stability and a high mechanical load capacity as well as little abrasive wear. Finally PVC exhibits good tolerance in the medical field and therefore is also recommended for allergic persons. Due to its dipole moment of the C—Cl bond in the polymer, PVC can be safely bonded to itself or other materials by means of high-frequency engineering.

The PUR material shows various physico-chemical advantages and PUR already represents a material which has been widely used and accepted in the medical domain.

The PTFE and FEP materials show the advantage that they have favorable inherent sliding properties and are highly resistant to environmental influences.

In accordance with a further development, at least one surface of the tube is functionalized by negatively charged, positively charged, polar or non-polar groups, wherein it is especially preferred that the surface is hydrophilic.

In an embodiment of the invention at least one outer face or surface of the tube is treated in such manner that the surface is functionalized in a desired way. Especially functionalizing can take place in a way that the hydrophilicity, lipophilicity and/or the surface tension of the surface are adjusted. It is possible by adjusting the hydrophilicity and/or lipophilicity to bind hydrophilic or lipophilic lubricants more strongly to the surface by appropriate interactions and thus to further reduce friction. In an especially preferred embodiment, after adjusting an increased hydrophilicity (“hydrophiling”) water or aqueous solutions, such as e.g. physiological saline, or aqueous solutions or dispersions of lubricants, such as polyethylene glycol (PEG), cellulose or algae-based lubricants, can be used as lubricants.

Preferably the functionalizing is carried out according to the methods described in the international patent applications WO-A-00/26180, WO-A-2006/075183 and WO-A-2008/023170, wherein the surface of a substrate is contacted with a carbene precursor, a reactive carbene intermediate is produced from the carbene precursor so that it reacts with the substrate for functionalizing the surface and the activated substrate is functionalized by introducing chemical groups having desired physical or chemical properties. The reactive carbene intermediate is preferably produced from the carbene precursor under conditions resulting in an irreversible bonding with the substrate (i.e. the tube surface). This “curing” can be brought about e.g. by heating, light radiation and/or ultrasonic waves.

The increased hydrophilicity of the surfaces and/or the better wettability thereof with aqueous mediums by a surface modification can preferably be brought about by introducing charged groups (e.g. acidic groups) or introducing polar groups (e.g. alcohols, amines). In this way, it is possible to further improve the sliding properties of the surface. In the same way, i.e. by introducing appropriate lipophilic groups, also the lipophilicity and the bonding of lipophilic lubricants can be increased.

In a preferred embodiment, the tube according to the invention is an eversion tube or an endoscope shaft or an endoscope shaft cover.

The tube according to the invention may also be a single-layer tube which exhibits improved properties by the afore-described adjustment of the Shore hardness, the afore-described appropriate functionalizing of at least one surface and/or the afore-described coating of at least one surface. In the event of a single-layer tube made of one of the afore-mentioned thermoplastic materials, the Shore hardness preferably is within the range of 50 to 65 and the wall thickness of the tube preferably is less than 1.6 mm.

In accordance with another development, the layers of the polymer tube have a different thickness. In accordance with another development, the Shore hardness of at least one layer is higher than the Shore hardness of a layer thicker compared to that. Preferably the Shore hardness of at least one layer is within the range of 50 to 65, preferably of 55 to 60 and the Shore hardness of at least one layer is within the range of 70-96.

In accordance with another development, after eversion a thinner and/or harder layer of the eversion tube forms the inner surface of the tube and a thicker and/or softer layer forms the outer surface of the tube, the latter layer being directly in contact with the patient.

This structure of the tube permits to obtain the desired mechanical strength of the tube.

Especially the use of a thin hard layer in combination with a thick soft layer in the afore-mentioned order is especially preferred for use in an eversion tube, as the hard thin layer supports the propulsion of the tube, while the soft layer cladding the intestinal wall contributes to a smaller bending radius and thus avoids or reduces mechanical irritations inside the patient, e.g. of the intestinal wall.

According to another further development, to at least one surface of the tube a coating is applied which is preferably selected from polyamide (PA), polyethylene (PE) or from perfluoroethylene propylene copolymer (FEP). For better adhesion of this/these coating(s) an adhesive layer may be provided between the surface and the coating, where appropriate.

Applying such a coating of substances having good sliding properties such as PA, PE and/or FEP further reduces undesired friction.

In accordance with another development, a lubricant is compounded into the tube material, the lubricant being preferably selected from glycerol, silicone and/or paraffin oil, synthetic waxes, polyethylene, polyethylene glycol (PEG) and other biocompatible substances having lubricating properties. Preferably during manufacture the lubricant is compounded into the tube material, i.e. mixed into the molten polymer mass prior to extrusion (“compounding” as it is called). Introducing the lubricant in this way entails a particularly advantageous lubricating effect, i.e. the reduction of friction, wherein this introduction into the material makes an additional step of applying a lubricant redundant.

According to another development, the tube has a preferred inner diameter of 10.4 to 12 mm and a preferred wall thickness of 0.5 to 1.6 mm.

According to another development, preferably at least one layer has a thickness of 0.5 to 1.3 mm and/or a layer has a thickness of 0.02 to 0.3 mm.

According to another development, the polymer tube is an eversion tube.

According to a preferred embodiment of the invention, the medico-technical flexible polymer tube comprises at least two layers.

Hereinafter the term “inner layer” means that it is a layer of the tube which has no contact to the surrounding, i.e. at least one further layer is provided on each side of an inner layer. The term “outer layer” relates to layers having direct contact to the surrounding, wherein the “external outer layer” is provided on the outer surface of the tube, while the “internal outer layer” is provided on the inner surface of the tube, i.e. is surrounded by the tube wall.

The layers of the multi-layer medico-technical polymer tube preferably have different thicknesses, wherein it is further preferred that the Shore hardness of a thinner layer is higher than the Shore hardness of a thicker layer of the tube.

The order and the number of layers are selected according to the intended use in each case.

In the case of an eversion tube, prior to everting the tube a thinner layer or a layer having the higher hardness may form an external outer layer of the tube. In the case of a two-layer structure, this entails that directly after manufacture the thin or harder layer forms the external outer layer of the tube (outer surface of the tube) and after everting the tube the thin and harder layer is provided inside and the thicker and softer layer contacts the intestinal surface of the patient. However, an inverted structure is also possible.

In the event of a tube having more than two layers, the two outer layers of the tube can be formed by thinner layers compared to the other layers of the tube or layers having a higher hardness compared to the other layers of the tube. The thicker layer(s) or the layer(s) having a lower hardness compared to the other layers of the tube can form an outer layer or inner layer of the tube.

The Shore hardness of the various layers can be adjusted in a preferred embodiment of the invention by the addition of softeners, as they are called. It is also possible, however, to adjust the Shore hardness of the tube layers without the use of softeners in response to the thermoplastic material used by appropriately selecting the manufacturing conditions of the thermoplastic or plastic material.

Preferably the softeners used are toxicologically harmless and dissolve out of the tube material only to a very small extent, i.e. they show practically no migration out of the tube material or between the different layers of the tube so that the tube can be stored for a quite long period of time (>1 year) and only extremely small amounts of softener get into the patient. Due to the short dwell time of the endoscope tube inside a patient, practically no migration of softener into the patient takes place.

Different concentrations of softener in different tube layers can possibly be compensated by the use of appropriately different softeners such that no significant compensation by diffusion occurs. In order to avoid a compensation of the softener concentration in the wall layers having a different Shore hardness, a separating layer may be provided between the different layers of the tube, if necessary.

Especially preferred are phthalate-free softeners containing alternatives to the previously common softeners such as dioctyl phthalic acid (DOP). Materials containing such softeners are also referred to as “NO DOP” materials. Softeners of this type are, e.g., diethylhexyl terephthalate (DEHTP), alykyl sulphonic acid ester (ASE), acetyltributyl citrate (ATBC), di-(2-ethylhexyl)adipate (DEHA; synonym: DOA) and acetylated castor oil derivative, but also the softeners described in the German patent specifications DE-T-60023 531, DE-A-101 41 250, DE-A-10 2004 029 135, DE-A-10 2004 036 202, DE-A-102 48 878. Especially preferred softeners are di-(isononyl)-cyclohexane-1,2-dicarboxylate (DINCH), tri-(2-ethylhexyl)trimeliate (TEHTM) or tri-2-ethylhexyltrimelitic acid ester (TOTM). But also combinations of one or more of these softeners can be used.

The amount of softener used depends on the desired Shore hardness, wherein the higher the share of softener, the lower the Shore hardness of the material. Especially preferred the softeners can be contained at concentrations of up to 50% by weight related to the total weight in the thermoplastic material used.

Further details on softeners to be employed and the concentration thereof, methods of adjusting the Shore hardness of plastic materials as well as suited additives can be taken e.g. from the “Taschenbuch der Kunststoff-Additive¹”; R. Gaechter and H. Mueller (publisher) 3rd Edition; Munich, Vienna, Hanser 1989 and “Kunststoff-Taschenbuch²” 16th Edition, Carl Hanser Verlag Munich. ¹Paperback of plastic additives²Plastics paperback

Furthermore, additives selected from antioxidants, fillers, UV adsorbing agents, stabilizers, chelating agents and pigments can be added to the thermoplastic material as required.

According to another development, at least one surface of the tube is provided with a lubricant. Preferably the lubricant is selected from water or water-based lubricants, polyethylene glycol (PEG), cellulose or algae-based lubricants, glycerol, paraffin oil, long-chain fatty alcohols, metallic soaps, fatty acid glycerol esters, polyethylene, synthetic waxes, agar-agar, plant oil, a fat-wax mixture, (e)PTFE sliding lacquer, (e)PTFE powder, graphite, talcum, various silicone coatings, silicone oil, graphite and/or Teflon powder dispersed in lubricating oil as well as (e)PTFE and/or glass spherules dispersed in lubricating oil and other biocompatible substances having lubricating properties, or a combination of two or more thereof. In the case of a hydrophilic or hydrophilized surface, water and water-based lubricants can be used. In the case of a non-functionalized surface of PVC, glycerol or paraffin oil and, in the case of a non-functionalized surface of PUR, silicone oil can be utilized.

A further especially preferred embodiment of the invention provides a medico-technical flexible polymer tube of thermoplastic material having at least two layers, the layers differing at least by their Shore hardness as described in the foregoing and at least one outer face or surface of the tube being functionalized as afore described.

The afore-described aspects, further developments and embodiments can be freely combined, wherein each measure per se entails an improvement, but synergy effects are resulting from the combination.

In accordance with another development of the invention, a method of manufacturing a medico-technical flexible polymer tube of thermoplastic material is provided, the polymer tube being manufactured in several layers of thermoplastics having a different Shore hardness and/or at least one surface of the polymer tube being subjected to a treatment reducing the friction.

The manufacturing method of the medico-technical polymer tube should be as simple as possible, because such polymer tube possibly is a throwaway article and thus the manufacture thereof should be inexpensive. Moreover, the method should be easily adapted to different tube materials.

Further, it is desirable that the method provides a tube which can be easily attached to or provided with further materials such as supplementary lubricants. It is another object of the invention to provide a corresponding polymer tube as such the sliding properties of which are improved as described above, which is inexpensive and can be excellently combined with different further lubricants.

In accordance with another development, in the method according to the invention one or more thermoplastic materials are co-extruded into several layers.

In accordance with another development, in the method according to the invention at least one layer of the polymer tube is made of PVC, PUR, FEP, PA, PE or PTFE.

In accordance with another development, in the method according to the invention at least one surface of the tube is functionalized, wherein at least one surface of the tube is functionalized by introducing and/or applying negatively charged, positively charged, polar or non-polar groups, wherein preferably at least one surface of the tube is hydrophiled.

In accordance with another development, in the method according to the invention at least one surface of the tube is preferably functionalized by the following steps of:
a) contacting the tube surface by a carbene precursor;
b) generating a reactive carbene intermediate from the carbene precursor so that it reacts with the material of the surface for functionalizing the surface; and
c) further functionalizing the activated surface obtained by introducing chemical groups having desired physical and/or chemical properties.

The carbene precursor is preferably applied by immersion and/or spray coating.

In accordance with another development, the Shore hardness of the layers of the polymer tube and/or the thickness of the layers are differently adjusted in the method according to the invention, wherein the Shore hardness of at least one layer is adjusted to be higher than the Shore hardness of at least one layer which is thicker compared thereto. Preferably the Shore hardness of at least one softer layer is adjusted within the range of 50 to 65, preferably of 55 to 60, and that of at least one harder layer is adjusted within the range of 70 to 96.

According to another development, in the method according to the invention a coating preferably selected from polyamide (PA), polyethylene (PE) or of perfluoroethylene propylene copolymer (FEP) is applied to at least one surface of the tube, an adhesive layer being provided between the surface and the coating, where appropriate. The coating can be applied by extrusion, bonding, immersion and/or spray coating.

According to another development, the inner diameter of the tube is preferably adjusted to 10.4 to 12 mm.

According to another development, the wall thickness of the tube is adjusted to 0.5 to 1.6 mm.

According to another development, the thickness of at least one layer is adjusted to 0.5 to 1.3 mm.

According to another development, the thickness of at least one layer is adjusted to 0.02 to 0.3 mm.

According to another development, a lubricant is compounded into at least one thermoplastic material.

According to another development, for compounding the lubricant is selected from glycerol, silicone and/or paraffin oil, synthetic waxes, polyethylene, polyethylene glycol (PEG) and other biocompatible substances having lubricating properties.

In accordance with another development, in addition a lubricant is provided on at least one surface of the tube.

Shaping the plastic material in tubular form when manufacturing the tube in the method according to the invention can be performed by plastics processing techniques known per se. According to the invention, an extrusion process is preferred, as possibly desired specific cross-sectional shapes and/or profiles can be imparted to the tube in this way. During such an extrusion process a profiled surface having e.g. nap-shaped projections can be formed on at least one tube surface. “Tube surface” in this context is both the outer surface and the inner (cover) surface of the tube. For manufacturing the medico-technical polymer tube according to the invention preferably an extrusion method is employed, wherein it is especially preferred that the at least two layers of the multi-layer tube are jointly extruded, i.e. co-extruded.

Preferably an adhesion is provided between the individual layers of the tube by co-extrusion. Yet this can equally be brought about by adhesion promoters or surface treatments suited for the respective plastics. In the case of PVC a safe connection of the layers is possible due to the dipolar moment of the C—Cl bond in the PVC polymer by means of high-frequency engineering.

During manufacture a lubricant, preferably glycerol, silicone and/or paraffin oil, synthetic waxes, polyethylene, polyethylene glycol (PEG) or other biocompatible lubricants can be compounded into the tube material, i.e. mixed into the molten polymer mass prior to extrusion (“compounding” as it is called). This introduction of lubricant entails an especially advantageous lubricating effect, i.e. the reduction of friction, wherein this introduction into the material may make an additional step of applying a lubricant redundant.

In the manufacturing method according to the invention of the plastic tubes according to the invention, either during the extrusion process itself or after the extrusion process a profile, e.g. in the form of naps, can be produced on at least one tube surface in a separate step. The profiled surface in the form of a nap produced during the extrusion process preferably is, for instance, a continuous spirally circumferential projection.

In a preferred embodiment of the invention, at least one of the surfaces of the eversion tube is provided with a coating of a polyolefin, preferably polyethylene (PE). This has the advantage that surfaces of polyolefins, preferably polyethylene, have a good sliding capacity without the addition of further lubricants.

In another preferred embodiment of the invention, a coating of a fluoroelastomer is applied to at least one surface of the tube, perfluoroethylene propylene copolymer (FEP) being especially preferred.

In a further preferred embodiment of the invention, a coating of polyamide (PA) is applied to at least one surface of the tube.

Since polyolefins, fluoroelastomers or polyamide possibly may only have a low inherent adhesion on other thermoplastic materials, in the case of use of polyolefins and fluoroelastomers or polyamide it may be necessary to promote or increase the adhesion by the use of adhesion promoters or adhesives, e.g. low-molecular polyolefins having acidic groups at one end.

Also regarding the method of the invention, the afore-described aspects, developments or embodiments can be freely combined to one another, wherein each measure per se entails an improvement, but from the combination synergy effects are resulting.

Furthermore, in accordance with the invention, a medico-technical polymer tube is provided which is obtained by the method according to the invention.

The present invention moreover provides an endoscope or coloscope comprising an eversion tube drive, the endoscope (and the endoscope shaft or an endoscope shaft cover put thereon) and/or the eversion tube preferably consist of the afore-described plastic material.

The eversion tube unwinds on the endoscope shaft cover. It is moreover preferred that a lubricant is provided between and/or in the nap-shaped projections. It is preferred in this context that the nap-shaped projections are perforated lubricant reservoirs. In a preferred embodiment, the nap-shaped projections are formed of spherules of (e)PTFE or glass. The preferred spherule diameter is within the range of up to 0.2 mm. The use of such spherules effectuates a further reduction of friction which entails definitely improved sliding properties both with a tube-to-tube friction and with a tube-to-shaft friction. One reason of this improvement is a definitely reduced bearing surface, namely only the tangential area of the spherules. Moreover, the spherules serve as spacers between which further lubricant can be filled. In this respect, as to their effect the spherules correspond to the afore-mentioned naps.

Especially when the medico-technical polymer tube of the present invention is used as eversion tube, a thermoplastic material as described above is suited especially well as basic substance of such tube, because it is comparatively inexpensive and can be easily treated such that the desired improvement can be achieved.

When use is made of the medico-technical polymer tube of the present invention as endoscope shaft cover, the afore-described thermoplastic materials are excellently suited. Hereby a good connection to the actual shaft can be obtained while simultaneously the friction is reduced vis-a-vis the outside of the eversion tube.

In a preferred embodiment the eversion tube and/or the coloscope is provided in an opaque packaging to prevent premature ageing of the materials used.

As additional lubricants which are applied to the tube surface(s) the afore-mentioned lubricants as well as especially non-hydrated or partly to completely hydrated vegetable oils such as rape-seed oil and sunflower oil are taken into account. Also a wax-containing fat or fat mixture is suited particularly well, for instance oil or oil mixture, preferably a mixture of vegetable oil and wax.

It has turned out that the use of a 50:50 mixture of completely hydrated rape-seed oil and highly oleic-acid containing sunflower oil (HO sunflower oil, as it is called) having a solidification point within the range of preferably 25 to 50° C., more preferably 30 to 40° C. and especially preferred 35 to 39° C. as well as a clear melting point of preferably 50 to 60° C. and more preferably 54 to 56° C., exhibiting very good sliding properties and being adapted to be used in endoscopic application with good tolerance. In order to even further improve the adhesion of such mixture to the polymer tube (either the polymer tube as such or the polymer tube having nap-shaped projections), preferably a particular amount of wax is added to the mixture or already contained in the same, for instance in non-winterized oil (non-fractioned oil).

An afore-described fat, fat mixture, oil or oil mixture can also be introduced into the plastic material itself, preferably into the plastic material of thermoplastic, especially that of thermoplastic polyurethane.

When extruding the material for forming the tube according to the invention, a spirally circumferential nap (projection) continuous on the tube surface can be formed especially well.

It is further possible to produce the naps in a separate step following the extrusion, for instance by cutting, punching, stamping, laser engraving or the like. In these operations, usually the material remaining between the naps is abraded. However, the naps can also be produced, by a separate step, out of a smooth surface, for instance by foaming specific surface areas, e.g. by local, e.g. point-shaped, heating of the surface (for example by a laser).

Such nap-shaped projections (or naps), e.g. a spiral nap, on the surface serve as spacers on the tube surface, with a lubricant being adapted to be filled between the spacers.

It is also possible that the lubricant is provided alternatively or in addition in the naps themselves which then serve as lubricant reservoir. The lubricant can escape from said lubricant reservoirs when pressure is applied, for instance.

The tube manufactured by the afore-described method already as such is suited very well for medico-technical applications, especially in endoscopy. In order to achieve a further improvement of the sliding properties, it is frequently advantageous to apply a further lubricant to the tube surfaces exposed to friction.

The endoscope described in the present invention includes an eversion tube drive, wherein the endoscope (i.e. the endoscope shaft or an endoscope shaft cover put thereon) and/or the eversion tube consist of a plastic material which is preferably selected from the afore-mentioned ones.

As stated in the foregoing, the method according to the invention is especially directed to manufacturing a polymer tube forming a shaft cover for an endoscope or an eversion tube for an endoscope. Accordingly, the present invention also includes a medico-technical polymer tube which is obtained by the method according to the invention.

### EXAMPLES

Hereinafter the invention is explained by way of examples, wherein these examples serve for the purpose of illustration and shall not be interpreted in a restrictive manner.

### 1st Example

The following variants of a single- or multi-layer eversion tube for an endoscope are manufactured of PVC by extrusion:
a) A single-layer tube of PVC (Shore hardness 55) having a wall thickness of 0.9 mm and an inner diameter of 10.5 mm is extruded by means of an extruder. The internal outer layer or inner surface formed directly after extrusion (i.e. the outer surface after eversion) is provided with an adhesive layer of low-molecular polyolefins having acidic groups at one end and a layer of PE having a thickness of 0.05 mm is applied. In another variant of this example both outer layers of the tube are coated with PE.
b) A two-layer tube of PVC having an outer layer (Shore hardness 90; thickness 0.1 mm), an inner layer (Shore hardness 55; thickness 0.8 mm) and having a wall thickness of 0.9 mm as well as an inner diameter of 10.5 mm is extruded by means of an extruder. The inner surface formed directly after extrusion (i.e. the outer surface after eversion) is provided with an adhesive layer of low-molecular polyolefins having acidic groups at one end and a layer of PE is applied at a thickness of 0.05 mm. In another variant of this example both outer layers of the tube are coated with PE.

### 2nd Example

Tubes of PVC are manufactured in accordance with the conditions of example 1, wherein instead of a PE coating a coating of FEP at a thickness of 0.1 mm is applied to one or both outer layers (surfaces) of the eversion tube.

### 3rd Example

Two variants of tubes of PVC are manufactured in accordance with the conditions of example 1, wherein instead of a PE coating the surface has been functionalized by hydrophilic groups utilizing carbene precursors.

### 4th Example

A two-layer co-extruded tube having a layer of PVC (Shore hardness 60) and a layer of PUR (Shore hardness 70) is extruded according to the conditions and variants of the examples 1 to 3 in a way that directly after extrusion the PVC layer forms the outer layer of the tube. The two outer layers of the extruded two-layer tube have been coated or functionalized according to the examples 1 to 3.

### 5th Example

A single-layer tube of PTFE (Shore hardness 65) and having a wall thickness of 0.8 mm as well as an inner diameter of 10.5 mm is extruded by means of an extruder.

### 6th Example

The following variants of a single- or multi-layer eversion tube for an endoscope are manufactured of PUR by extrusion:
a) A single-layer tube of PUR (Shore hardness 65) and having a wall thickness of 0.7 mm as well as an inner diameter of 11.1 mm is extruded by means of an extruder. A lubricant (e.g. glycerol, paraffin oil, silicone oil, synthetic waxes, polyethylene, polyethylene glycol (PEG)) are compounded into the PUR, wherein a smooth oily-lubricating surface is produced which can dispense with a coating, possibly also with the addition of a lubricant.
b) A two-layer tube of PUR having an outer layer (Shore hardness 90; thickness 0.12 mm) and having an inner layer (Shore hardness 50; thickness 0.8 mm) and a wall thickness of 0.92 mm as well as an inner diameter of 11.1 mm is extruded by means of an extruder. The inner surface formed directly after extrusion (i.e. the outer surface contacting the patient after eversion) is subjected to a coating with PE or FEP as in the preceding example or to a functionalization of the surface by hydrophilic groups utilizing carbene precursors.

1. A medico-technical flexible polymer tube of a thermoplastic or of thermoplastics, wherein
a) the polymer tube includes at least two layers having a different Shore hardness; and/or
b) the polymer tube includes one or more layers and at least one surface of the polymer tube is functionalized.

2. A medico-technical flexible polymer tube according to claim 1, wherein the polymer tube is an eversion tube or an endoscope shaft cover.

3. A medico-technical flexible polymer tube according to claim 1, wherein at least one layer of the polymer tube is made of PVC, PUR, FEP, PE, PA or PTFE.

4. A medico-technical flexible polymer tube according to claim 1, wherein at least one surface of the tube is functionalized by negatively charged, positively charged, polar or non-polar groups.

5. A medico-technical flexible polymer tube according to claim 1, wherein at least one surface of the tube is hydrophilic.

6. A medico-technical flexible polymer tube according to claim 1, wherein the layers have a different thickness.

7. A medico-technical flexible polymer tube according to claim 6, wherein the Shore hardness of at least one layer is higher than the Shore hardness of a layer thicker compared thereto.

8. A medico-technical flexible polymer tube according to claim 1, wherein the Shore hardness of at least one layer is within the range of 50 to 65, preferably of 55 to 60.

9. A medico-technical flexible polymer tube according to claim 1, wherein the Shore hardness of at least one layer is within the range of 70 to 96.

10. A medico-technical flexible polymer tube according to claim 1, wherein
a) in a tube including two layers a thinner and/or harder layer of the tube forms the internal outer layer of the tube and a thicker and/or softer layer forms the external outer layer of the tube; or
b) in a tube including three layers a respective thinner and/or harder layer of the tube forms each of the internal and external outer layers of the tube and at least one thicker and/or softer layer forms an inner layer of the tube.

11. A medico-technical flexible polymer tube according to claim 1, wherein a coating preferably selected from polyamide (PA), polyethylene (PE) or from perfluoroethylene propylene copolymer (FEP) is applied to at least one surface of the tube.

12. A medico-technical flexible polymer tube according to claim 11, wherein an adhesive layer is provided between the surface and the coating.

13. A medico-technical flexible polymer tube according to claim 1, wherein a lubricant is compounded into at least one thermoplastic material.

14. A medico-technical flexible polymer tube according to claim 13, wherein the lubricant is selected from glycerol, silicone and/or paraffin oil, synthetic waxes, polyethylene, polyethylene glycol (PEG) and other biocompatible substances having lubricating and sliding properties or from a combination of two or more thereof.

15. A medico-technical flexible polymer tube according to claim 1, wherein at least one surface of the tube is provided with a lubricant.

16. A medico-technical flexible polymer tube according to claim 15, wherein the lubricant is selected from water or water-based lubricants, polyethylene glycol (PEG), cellulose or algae-based lubricants, glycerol, paraffin oil, higher fatty alcohols, metallic soaps, fatty acid glycerol esters, polyethylene, synthetic waxes, agar-agar, vegetable oil, a fat-wax mixture, (e)PTFE sliding lacquer, (e)PTFE powder, graphite, talcum, various silicone coatings, silicone oil, graphite and/or Teflon powder dispersed in lubricating oil as well as (e)PTFE and/or glass spherules dispersed in lubricating oil and other biocompatible substances having lubricating and/or sliding properties, or a combination of two or more thereof.

17. A medico-technical flexible polymer tube according to claim 1, wherein the tube has an inner diameter of 10.4 to 12 mm.

18. A medico-technical flexible polymer tube according to claim 1, wherein the tube has a wall thickness of 0.5 to 1.6 mm.

19. A medico-technical flexible polymer tube according to claim 1, wherein at least one layer has a thickness of 0.5 to 1.3 mm.

20. A medico-technical flexible polymer tube according to claim 1, wherein at least one layer has a thickness of 0.02 to 0.3 mm.

21. A medico-technical flexible polymer tube made of thermoplastic material, wherein
a) the polymer tube consists of a layer having a Shore hardness of 50 to 65, and/or
b) at least one surface of the polymer tube is functionalized.

22. A medico-technical flexible polymer tube according to claim 21, wherein the tube has a wall thickness that is smaller than 1.6 mm.

23. (canceled)

24. A method of manufacturing a medico-technical flexible polymer tube made of thermoplastic or of thermoplastics, wherein
a) the polymer tube is manufactured of several layers of thermoplastic materials having a different Shore hardness, and/or
b) at least one surface of the polymer tube is functionalized by one or more layers of thermoplastic material.

25. A method according to claim 24, wherein one or more thermoplastic materials are co-extruded in several layers.

26. A method according to claim 24, wherein at least one layer is made of PVC, PUR, FEP, PE, PA and/or PTFE.

27. A method according to claim 24, wherein at least one surface of the tube is functionalized by introducing and/or applying negatively charged, positively charged, polar or non-polar groups.

28. A method according to claim 24, wherein at least one surface of the tube is hydrophiled.

29. A method according to claim 24, wherein functionalizing is performed by the following steps of:
a) contacting the tube surface with a carbene precursor;
b) producing a reactive carbene intermediate of the carbene precursor so that it reacts with the material of the surface for functionalizing the surface; and
c) further functionalizing the activated surface obtained in step b) by introducing chemical groups having desired physical and/or chemical properties.

30. A method according to claim 29, wherein the carbene precursor is applied by immersion or spray coating.

31. A method according to claim 24, wherein the Shore hardness of the layers is differently adjusted.

32. A method according to claim 24, wherein the thickness of the layers is differently adjusted.

33. A method according to claim 24, wherein the Shore hardness of at least one layer is adjusted to be higher than the Shore hardness of at least one layer thicker compared thereto.

34. A method according to claim 24, wherein the Shore hardness of at least one softer layer is adjusted within the range of 50 to 65, preferably of 55 to 60.

35. A method according to claim 24, wherein the Shore hardness of at least one harder layer is adjusted within the range of 70 to 96.

36. A method according to claim 24, wherein a coating preferably selected from polyamide (PA), polyethylene (PE) or of perfluoroethylene propylene copolymer (FEP) is applied to at least one surface of the tube.

37. A method according to claim 36, wherein the coating is applied by extrusion, bonding, immersion and/or spray coating.

38. A method according to claim 36, wherein an adhesive layer is provided between the surface and the coating.

39. A method according to claim 24, wherein the inner diameter of the tube is adjusted to 10.4 to 12 mm.

40. A method according to claim 24, wherein the tube has a wall thickness that is adjusted to 0.5 to 16 mm.

41. A method according to claim 24, wherein the thickness of at least one layer is adjusted to 0.5 to 1.3 mm.

42. A method according to claim 24, wherein the thickness of at least one layer is adjusted to 0.02 to 0.3 mm.

43. A method according to claim 24, wherein a lubricant is compounded into at least one thermoplastic material.

44. A method according to claim 43, wherein the lubricant is selected from glycerol, silicone and/or paraffin oil, synthetic waxes, polyethylene, polyethylene glycol (PEG) and other biocompatible substances having lubricating and/or sliding properties or from a combination of two or more thereof.

45. A method according to claim 24, wherein at least one surface of the tube is provided with a lubricant.

46. A method according to claim 45, wherein the lubricant is selected from water or water-based lubricants, polyethylene glycol (PEG), cellulose or algae-based lubricants, glycerol, paraffin oil, higher fatty alcohols, metallic soaps, fatty acid glycerol esters, polyethylene, synthetic waxes, agar-agar, vegetable oil, a fat-wax mixture, (e)PTFE sliding lacquer, (e)PTFE powder, graphite, talcum, various silicone coatings, silicone oil, graphite and/or Teflon powder dispersed in lubricating oil as well as (e)PTFE and/or glass spherules dispersed in lubricating oil and other biocompatible substances having lubricating and/or sliding properties, or a combination of two or more thereof.

47. An endoscope comprising;
a medico-technical flexible polymer tube of a thermoplastic or of thermoplastics, wherein
a) the polymer tube includes at least two layers having a different Shore hardness; and/or
b) the polymer tube includes one or more layers and at least one surface of the polymer tube is functionalized.
